(19) United States
(12) Patent Application Publication
(10) Pub. No.: US 2026/0263741 A1
(43) Pub. Date: Sep. 10, 2026

(54) CATHETER SYSTEM HAVING SEALING SYSTEM FOR PREVENTING BACK-FLOW WITHIN CEREBRAL VESSELS

(71) Applicant: MG-MMA INC., Calgary (CA)

(72) Inventors: Mayank GOYAL, Calgary (CA); Johanna OSPEL, Calgary (CA)

(21) Appl. No.: 19/165,669

(22) PCT Filed: Mar. 13, 2024
(86) PCT No.: PCT/CA2024/050304

§ 371 Date: Sep. 16, 2025

Related U.S. Application Data
(60) Provisional application No. 63/452,998, filed on Mar. 17, 2023.

(51) Int. Cl. A61M 25/00; A61M 25/01

(52) U.S. Cl. CPC A61M 25/0021; A61M 25/0043; A61M 25/0068; A61M 25/0108; A61M 2025/0042; A61M 2202/0007; A61M 2205/32; A61M 2210/12

(57) ABSTRACT

Catheter systems for treatment of chronic subdural hematoma (CSDH) are described. The catheter systems are configured to enable passage through small and tortuous vessels within the vasculature and include a sealing system that engages within a vessel that prevent back-flow of polymer agents within vessels when delivering polymer agents to affected vessels.

### FIELD

Catheter systems for treatment of chronic subdural hematoma (CSDH) are described. The catheter systems include a sealing system that engages within a vessel that prevent back-flow of polymer agents within the vessels when delivering polymer agents to affected vessels.

### BACKGROUND

Chronic subdural hematoma (CSDH) is a common neurosurgical condition characterized as an old clot of blood on the surface of the brain beneath the outer covering. These clots most often occur in patients over the age of 60 who have brain atrophy from shrinking or wasting of brain tissue due to age or disease. When the brain shrinks over time within the skull, it has been traditionally understood that minor head trauma can cause tearing of blood vessels over the brain's surfaces, resulting in a slow accumulation of blood. In an atrophied brain, the clots can become quite large before symptoms occur. Many patients cannot recall any particular traumatic event that may have caused the CSDH.

The incidence of CSDH has been reported as between 1.72 to 20.6 per 100,000 persons per year. The incidence increases progressively with age and is increased by a factor of 3.5 over the age of 70 and 6.5 over the age of 80.

The most common complaint is headache but also includes lethargy, memory impairment, confusion, weakness, nausea, vomiting, impaired vision and seizures.

CSDH is diagnosed via CT and MRI scans.

Current standards of care include surgically draining the CSDH. This can include drilling a hole in the skull (burr hole) or a craniotomy and the draining the blood mass through a catheter.

Unfortunately, the clinical course from this treatment is frequently complicated by recurrence with rates reported from 7.5%-29%. More than 90% of these recurrences happen within two months of primary surgery, and often lead to prolonged hospital stays and devastating outcomes in the elderly and frail population that is affected by CSDH. The treatment cost for recurrent CSDH is 132% higher than the treatment for non-recurrent CSDH.

### Chronic Inflammation as the Key Driver for CSDH

As noted above, the traditional theory was that head trauma leads to the disruption of bridging veins (acute SDH). Some of these acute hematomas were thought to persist and become CSDH.

However, recently it has become evident that an inflammatory response of the dural layers from trauma results in formation of new blood vessels (NBVs) with weak vessel walls that are prone to bleed recurrently and thereby cause CSDH. These new vessels mostly arise from one artery that is supplying the meningeal layers of the brain namely the middle meningeal artery as shown in FIG. 1.

### SUMMARY

In accordance with the disclosure, a catheter is described comprising: a catheter body having a distal catheter tip, the catheter body configured with a sealing system adjacent the catheter tip, the sealing system configured to enable contact with a vessel wall to effect sealing of distal fluids from proximal fluids on opposite sides of the sealing system and wherein the sealing system has sufficient compressibility to enable flexure of the sealing system around a tight bend of a patient's vasculature and to enable passage of an embolizing agent through the catheter body and from the distal catheter tip.

In various embodiments:
the sealing system includes a bulb having a bulb body, the bulb body having tapered distal surface.
the sealing system includes a bulb having a bulb body, the bulb body having tapered proximal surface.
the sealing system is a truncated bulb having a bulb body having a tapered distal surface and a truncated proximal surface.
the bulb body includes one or more slits, slots or channels.
the bulb body includes at least one circumferential, longitudinal or helical slit, slot or groove.
the sealing system includes an invertable conical cone having a conical leaf (referred to as an “umbrella”) and having a flexibility/stiffness enabling proximal inversion as the catheter body is distally advanced within a vessel and distal inversion when the catheter is moved proximally within a vessel.
the sealing system includes at least one outwardly extending disk from the catheter body.
the sealing system has an outer diameter of 0.8-1.7 mm.
the catheter body has an outer diameter of 0.5-1.0 mm.
the sealing system is 0-50 mm from the distal catheter tip.
the sealing system is 0-5 mm from the distal catheter tip.
the outer diameter of the sealing system is 1.5-2.5 times the OD of the catheter body.
the catheter includes a radio-opaque marker.

In another aspect, a kit comprising at least two catheters is described wherein each catheter has a sealing system having a different outside diameter.

In another aspect, a method of manufacturing a catheter is described comprising the steps of sliding a sealing system having an internal diameter appropriately sized to an outer diameter of the catheter body and adhering or gluing the sealing system to the catheter body.

### DESCRIPTION OF THE DRAWINGS

Aspects of the disclosure are described with reference to the drawings in which:

FIG. 1 is a schematic diagram of the middle meningeal artery (MMA) with its branches.

FIG. 2 is a schematic diagram of CSDH and two common surgical options including burr hole (A) and craniotomy (B).

FIG. 3 is a schematic diagram of middle meningeal artery (MMA) embolization showing (A) Chronic subdural hematoma, CSDH, with MMA supplying the covering of the brain and giving rise to many newly formed, curvy vessels (NBVs) with weak vessel walls that recurrently bleed and give rise to the CSDH. (B) shows a catheter delivering the embolic agents into the vessel. (C) shows, after few weeks, how the embolization, the pathological curvy vessels have diminished.

FIG. 3A is a schematic diagram showing the delivery of an embolic agent in accordance with the prior art.

FIG. 3B is a schematic diagram showing the delivery of an embolic agent through a catheter having a detachable tip in accordance with the prior art.

FIG. 4 is a schematic diagram of an MMA catheter in accordance with the present disclosure.

FIG. 4A is a schematic diagram of a bulb sealing system in accordance with one embodiment of the present disclosure having circumferential grooves.

FIG. 4B is a schematic diagram of a bulb sealing system in accordance with one embodiment of the present disclosure having a helical groove.

FIG. 4C is a schematic diagram of a bulb sealing system in accordance with one embodiment of the present disclosure having a truncated bulb and helical groove.

FIG. 4D is a schematic diagram of a bulb sealing system in accordance with one embodiment of the present disclosure having a truncated bulb and circumferential grooves.

FIG. 5 is a schematic diagram of an MMA catheter positioned within a tortuous vessel.

FIG. 6 is a schematic diagram of an MMA catheter having a sealing disks sealing system.

FIGS. 6A-6C are schematic diagrams of an MMA catheter having an invertable conical leaf sealing system where 6A shows the MMA catheter being advanced, 6B shows the step of inverting the conical leaf and 6C shows the conical leaf expanding against the vessel with the introduction of polymer into a vessel.

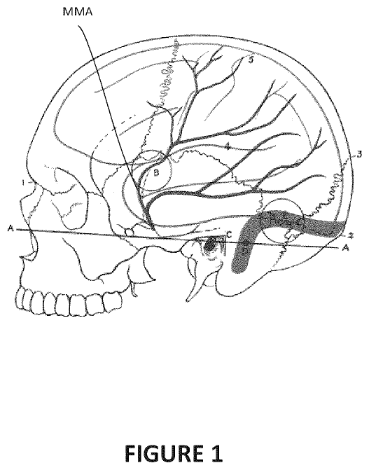

### DESCRIPTION

The inventors who have skill and experience in endovascular surgery have recognized problems in the treatment of CSDH and have developed catheter systems designed to improve the treatment of CSDH.

### Surgical Treatment

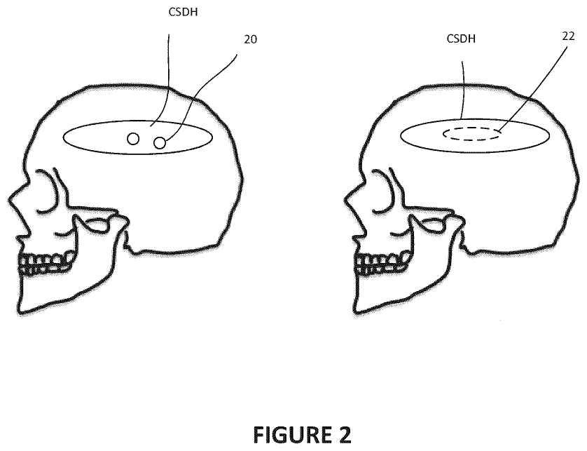

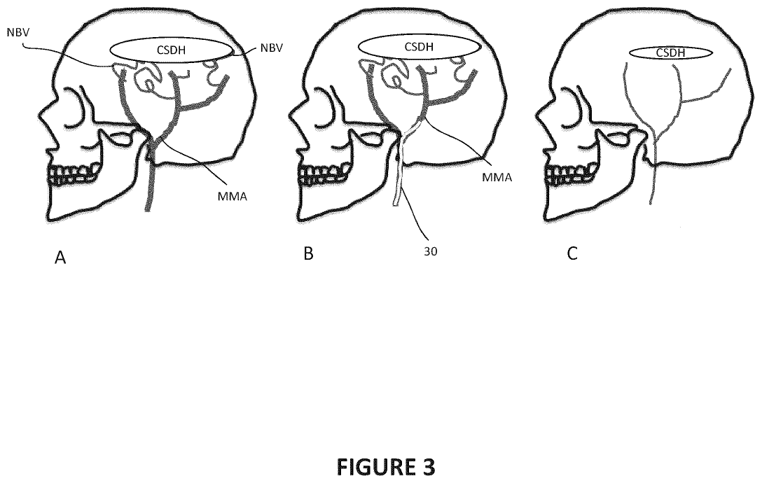

Currently, surgical drainage remains the mainstay of treatment for symptomatic CSDH. As shown in FIG. 2, CSDHs are commonly treated with burr hole 20 or craniotomy 22 surgery to enable drainage of the hematoma via a catheter. Surgical drainage is considered relatively straightforward. However, despite surgical treatment, many CSDH re-occur, with recurrence rates ranging between 7.5%-29%.

Importantly, surgery does not tackle any underlying inflammatory changes, and is therefore only a symptomatic treatment, and does not eliminate the cause of CSDH namely any underlying chronic inflammation. Surgery also often necessitates antiplatelet or anticoagulant therapy to avoid perioperative complications. As is known, the use of these agents further increases the risk of hematoma recurrence. Thus, surgical treatment is problematic in that it does not truly address the underlying cause of the CSDH.

### Middle Meningeal Artery Embolization

Embolization of the middle meningeal artery (MMA) has been used for the treatment of CSDH. MMA embolization is performed using tiny catheters through which liquid embolic agents (polymer agents; e.g. Onyx™) are injected to occlude the MMA and any new blood vessels (NBVs). When injected, these embolic agents enter the MMA and reduce the overall vascularity of the meninges including the new blood vessels that have formed due to chronic inflammation.

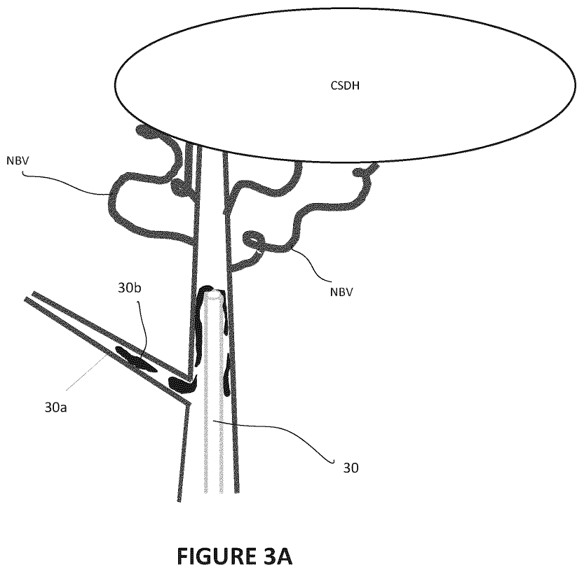

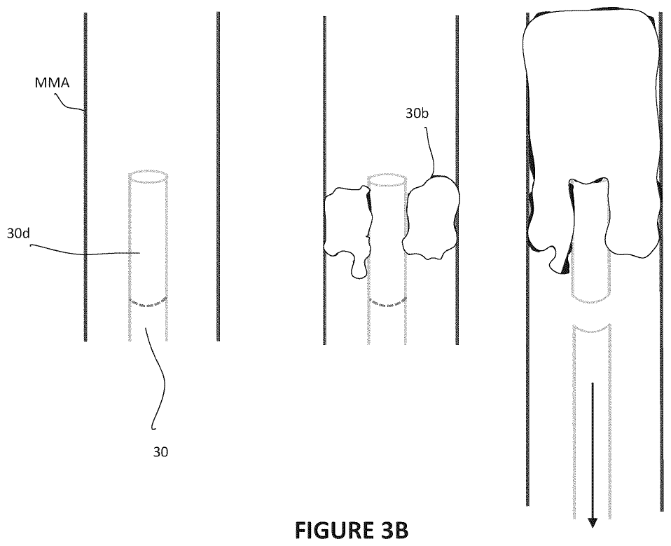

Occlusion of the MMA and newly formed vessels stops the blood flow in these vessels and effectively reduces the risk of bleeding and thus CSDH formation and recurrence as shown in FIG. 3.

FIG. 3 shows a schematic middle meningeal artery (MMA) embolization procedure where (A) shows a chronic subdural hematoma (CSDH) and NBVs prior to treatment (NBVs may or may not be seen with angiographic imaging). That is, a CSDH, with MMA supplying the covering may have given rise to many newly formed, curvy vessels (NBVs) with weak vessel walls that recurrently bleed and gave rise to the CSDH. (B) shows a catheter 30 delivering an embolic agent (e.g., Onyx™) into the MMA and NBVs. This results in vessel occlusion and shrinkage/disappearing of the NBVs over the course of a few weeks. (C) shows a few weeks after the embolization where the NBVs have disappeared, and the MMA has reduced. As a result, the CSDH has shrunk, and recurrence risk is substantially decreased.

MMA embolization has several challenges:
1) As shown in FIG. 3A, the MMA has anastomoses with important vessels 30a supplying the eyes and other important structures in the head including cranial nerves. Thus, the catheter 30 has to be navigated past the branch points at which the MMA gives rise to vessels supplying these other important structures. Importantly, reflux of the embolic agent 30b into these important branches has to be avoided.
2) As shown in FIG. 3B, in order to prevent backflow, a “plug” of embolic agent material 30b is created. To do this, the surgeon will administer the embolic agent in a manner such that a plug is formed around the catheter to “seal the catheter tip”. Forming this plug does require a little bit of backflow to wrap the embolic agent around the catheter tip. Also, forming the plug takes care and a relatively long time because a) the embolic agent material must be released slowly from the catheter and b) because it is viscous, it is slow flowing. Further, it takes a relatively long time (about 20 minutes) to harden and form a stable plug. Sometimes, a plug cannot be formed at all and/or the embolic agent continues to reflux instead of moving forward. This carries the risk of embolization of important MMA side branches (e.g. 30a; FIG. 3A). Additionally, even if embolization of important side branches does not occur, it leads to inadequate penetration of the distal vasculature resulting in an incomplete treatment. Since the distal vasculature is still patent, over a period of time, it can continue to get blood supply to alternative pathways.
3) As shown in FIG. 3B, if a plug can successfully be formed, the catheter tip often sticks to the plug and cannot be removed. Therefore, detachable catheters have been utilized, whereby the catheter has a detachment point below the tip, enabling the proximal portion of the catheter to be detached from the tip and withdrawn with the tip staying in the vessel once the procedure is finished. There are key challenges with this procedure including a. the surgeon typically allows reflux up to the detachment point which may not always be doable depending on the anatomy and the point of origin of critical branches just proximal to the tip of the microcatheter and b. it still adds to procedure time as the surgeon has to slowly inject to allow a controlled reflux to allow for a sufficient formation of a plug that can subsequently push the liquid embolic forward.
4) The MMA is very small and tortuous, and navigating a catheter in such a tiny vessel (often <1.5 mm) is challenging.
5) As noted above, the patients are often old and uncooperative and move a lot during the procedure, especially during prolonged procedures. Therefore, the procedure often has to be performed under general anesthesia, and the longer the anesthesia, the higher the risk of anesthesia-related side effects. Therefore, it is desirable to have the procedure as short as possible.
6) Vasospasm can occur during these procedures where the arterial vessels contract to narrow the vessel thus preventing catheters to be advanced through these vessels. Vasospasm is more likely to occur in patients who are smokers.

In other similar procedures in other areas of the body, in order to avoid back-flow or reflux, a catheter with a mounted balloon is often used. After positioning the catheter, the balloon in inflated to form a plug and force the liquid embolic agent forward. However, even the smallest balloon catheters are often not small enough to fit into a normal sized MMA which is common in most CSDH patients (i.e., one that is not supplying a fistula). Also balloon catheters are expensive.

In accordance with the disclosure, MMA catheters 10 enabling access into the MMA vessel system are described that provide effective sealing against a vessel wall to prevent back-flow whilst remaining sufficiently flexible to be navigated within the MMA and through tortuous regions of those vessels and that can be removed after release of embolic agent.

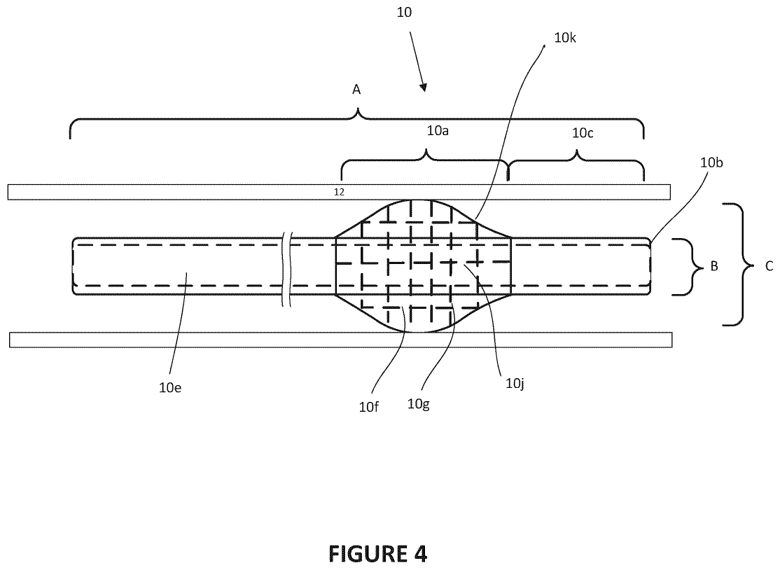

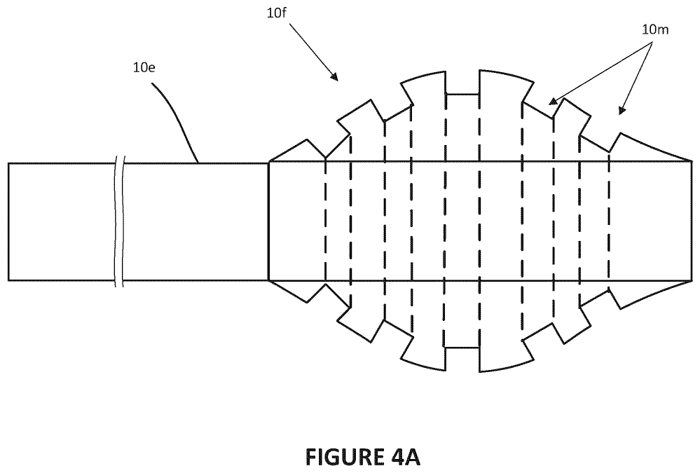

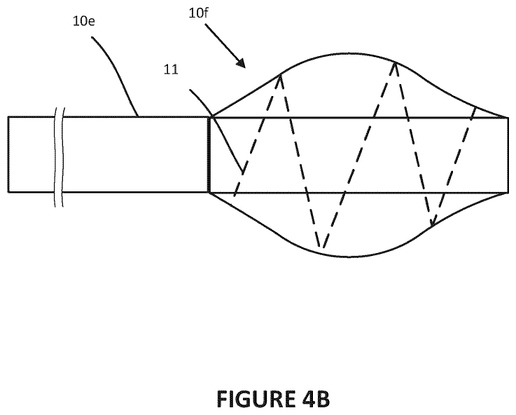

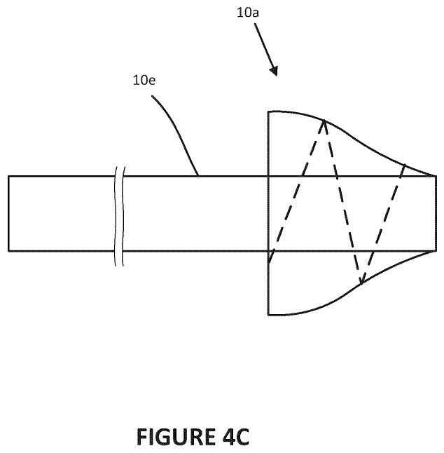

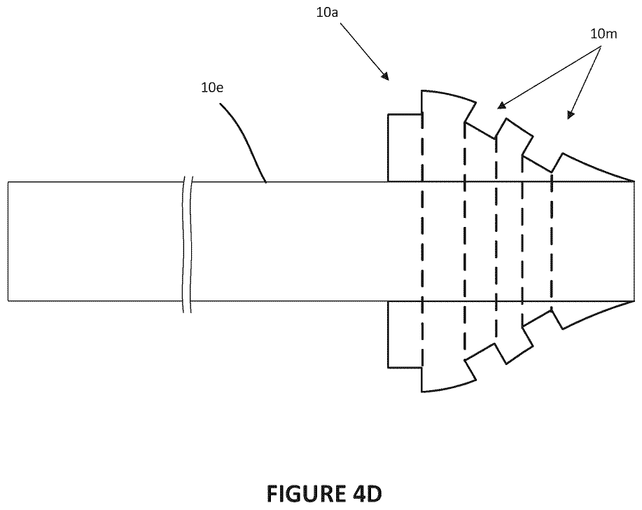

In general, and as shown in FIGS. 4-6, an MMA catheter 10 has a soft, highly flexible expanded region 10a at or close to the catheter tip 10b that can seal against a vessel 12. The expanded region described herein is defined as a sealing system. The portion of the catheter 10 between the catheter tip 10a and sealing system 10a is the tip region 10c. The sealing system 10a has the primary function of providing a seal between the MMA catheter 10 and the vessel wall 12 to prevent significant back-flow of polymer agents administered through the catheter. As opposed to a catheter with a detachable tip, where the tip remains in the vessel and blocks access for future procedures, the catheter described in the disclosure can be fully removed after the procedure, when it has been determined that there has been sufficient forward penetration of the embolic agent.

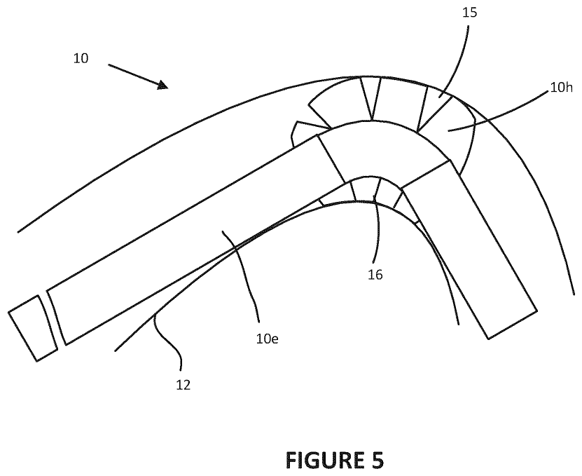

Furthermore, because it is not necessary to form a plug with the liquid embolic agent itself when using the MMA catheter 10, the procedure time can be substantially shortened, thus, complications due to patient movement can be reduced and general anesthesia may potentially be avoided. In various embodiments, the sealing system has the following properties:
defines an outer seal that can fully engage with an MMA vessel 12 to prevent back-flow of materials ejected from the tip of the MMA catheter.
When flexed within a tight vessel curve (often 120 degrees or more) is sufficiently expandable on an outer surface of the sealing system to enable the MMA catheter to flex around the tight vessel radius (FIG. 5).
When flexed within a tight vessel curve (often 120 degrees or more) is sufficiently compressible on an inner surface of the sealing system to enable the MMA catheter to flex around the tight vessel radius (FIG. 5).
The sealing system is fabricated from atraumatic materials to prevent injury to the vessel intima and/or minimize the risk of causing vasospasm.
The sealing system is positioned close to the tip of the MMA catheter approximately 0-50 mm from the tip, and more preferably 0-15 mm and still more preferably 0-5 mm from the tip 10b.
The OD of the sealing system is 0.8-1.7 mm being sized in 0.1 mm increments on appropriately-sized catheters enabling a surgeon to select an MMA catheter sized for a target vessel.
The outer diameter (OD) of the sealing system is approximately 1.5-2.5 times the OD of the catheter.
The sealing system may have a distally facing taper that facilitates forward movement of the sealing system within tight curves.
the sealing system is a forward-facing conical leaf having a stiffness enabling proximal inversion as the catheter is distally advanced and distal inversion when the catheter is moved proximally. The conical leaf is made of very soft material (typically silicon based).
the sealing system may be provided with radio-opaque markers to facilitate positioning.

Various features of the MMA catheter include:
The OD of the MMA catheter is 0.5-1.0 mm.
The MMA catheter has a proximal region 10e that is sufficiently long to extend to outside the body.
The overall length A of the MMA catheter is approximately 150-160 cm.

As shown in FIGS. 4-6, different embodiments of the MMA catheter 10 are shown. Generally, the MMA catheter has a lumen 10e and sealing system 10a.

As shown in FIG. 4, in one embodiment, the sealing system is a bulb 10f. The bulb may include one or more slits, slots or channels 10g (shown schematically in dotted lines) that enhance the ability of the bulb to navigate through a tight vessel as shown in FIG. 5. Such slits, slots or channels may be circumferential 10g, longitudinal 10j or helical 11 or other designs that provides the ability of the bulb to both expand and compress within a curve.

In one design, the bulb has a forward facing (distal) side of the bulb that may include a surface 10k that facilitates the deflection of the bulb around a curve to further minimize the risk of the bulb getting stuck within a tight curve. For example, the forward-facing surface 10k may include a concave surface that provides a shallower engagement angle when the bulb first engages with a curved vessel.

As shown in FIG. 4A, in one embodiment, the bulb may be provided with a series of circumferential grooves 10m formed in the outer surface of the bulb that provides space for compression and expansion of the bulb.

As shown in FIG. 4B, in one embodiment, the bulb may be provided with one or more helical grooves 11 formed in the outer surface of the bulb that provides space for compression and expansion of the bulb.

As shown in FIGS. 4C and 4D the sealing system 10a may be truncated on its proximal side.

In FIG. 5, the sealing system is shown in a tight curve where the bulb is shown compressed 16 on the inside portion of the curve and expanded 15 on the outside portion of the curve during this manoeuvre.

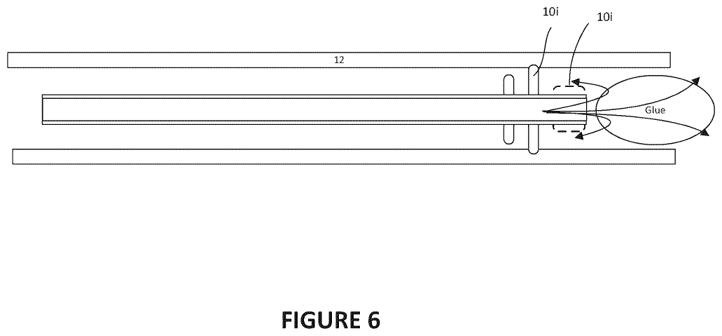

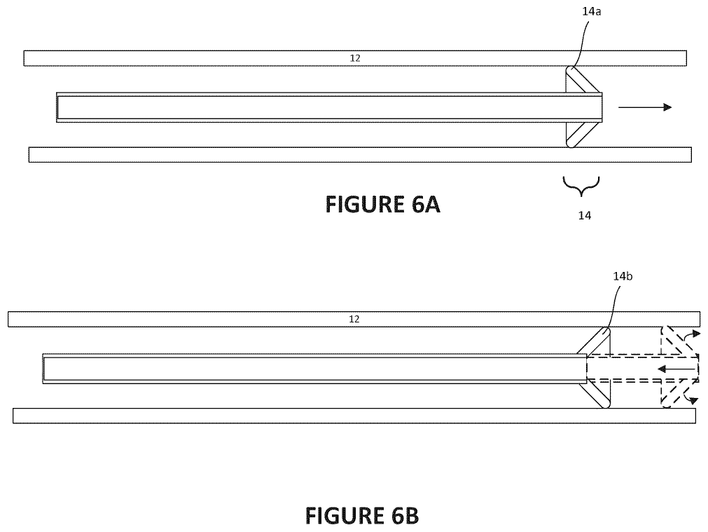

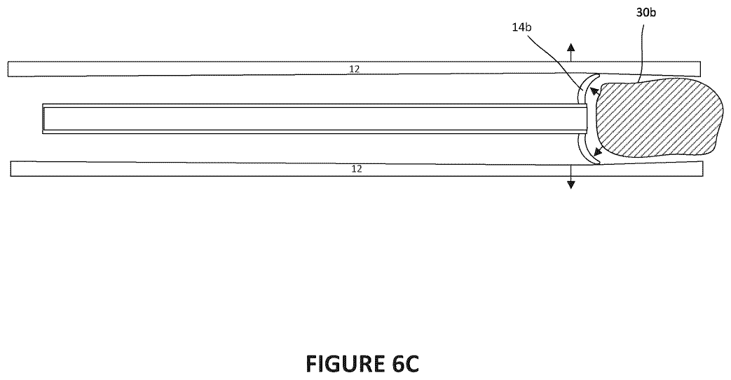

In FIG. 6, the sealing system is shown as series of separated disks 10i that may be distributed along a section of the MMA catheter. Such disks may be substantially adjacent to one another and may be of variable diameter with respect to one another. In one embodiment, the most distal and proximal disks may be smaller than the middle disk(s). During advancement, the disks may fold down against one another as the MMA catheter is pushed forward around curves but expand again when beyond a curve. The disks 10i may be of different diameters and profiles as shown in FIG. 6. For example, in FIG. 6, the two most proximal disks are wider than they are thick whereas the distal most disk is narrower than it is wide.

FIGS. 6A-6C illustrate the sealing system as a soft conical leaf 14 that can flip between a proximal inversion 14a (FIG. 6A) and a distal inversion 14b (FIG. 6B). In this embodiment, as the catheter is being pushed forward, the conical leaf may be proximally inverted and when the MMA catheter is in place, it is pulled back to place it in the distal inversion as shown by the arrow and dotted lines in FIG. 6B.

Furthermore, FIG. 6C shows the effect of injecting polymer. Normally, as polymer is injected, it increases the pressure within the vessel which may cause a slight expansion of the vessel as shown by the arrows in FIG. 6C. The increase in pressure may also cause the conical leaf 14b to flex proximally against the expanded vessel wall which may maintain sealing and minimize reflux of polymer.

Regardless of the particular style of sealing system, as described above, the sealing system is configured to the exterior of the MMA catheter. The sealing system may be configured to the exterior of the MMA catheter by various manufacturing techniques including being molded to the exterior of the catheter during casting of the catheter body or the sealing system may be produced as a separate component that would be positioned over the previously produced catheter and subsequently adhered/glued to the catheter body.

Importantly, the material of the sealing system is an atraumatic polymer (e.g. silicone) that is sufficiently soft to minimize the risk of inducing vasospasm yet with sufficient stiffness to provide a seal.

The catheter may be incorporated into a kit having two or more catheters each having a sealing system with different diameters to enable the surgeon to select a catheter most appropriate for the patient's vessels.

In use, the MMA catheter 10 is advanced over a wire that has been navigated to the MMA vessels. Upon positioning of the tip 10b (which may include radio-opaque markers), the wire is removed and the embolic/polymer agent may be delivered to the vessel as shown in FIGS. 6 and 6C. The sealing system prevents/minimizes back-flow of the polymer agent to the proximal side of the sealing system. Generally, during the procedure, the surgeon can determine the degree of forward penetration of the polymer agent due to the presence of radio-opaque particles in the polymer agent. When the surgeon is satisfied that forward penetration is sufficient, the MMA catheter can be removed without the formation of a plug around the catheter tip.

The MMA catheter is described above for use in the MMA vessels; however, it is understood that a similar system can be used in other small vessel procedures where the administration of polymer agents are required for treatment of similar conditions including brain and spinal arteriovenous fistula.

For this procedure, and for the purposes of description, an arteriovenous fistula is an abnormal connection between arteries (high flow high pressure vessels) and veins (low flow low pressure vessels). As an illustrative example, spinal veins normally drain blood from the spinal cord. If there is an abnormal connection between spinal arteries and veins, the pressure in the veins increases and flow reverses (abnormal backflow)—this results in blood congestion in the spinal cord and impedes the spinal cord function (for example, patients cannot walk any more and have disturbed bladder and bowel function). The abnormal veins also enlarge as a result and can be seen in the MRI.

The spinal cord congestion can be seen as an enlargement and hyperintense signal on the T2 weighted MRI.

Spinal arteriovenous fistulas can be cured by occluding the abnormal connection point between the spinal arteries and veins.

The problems are that a) spinal arteries are very small (1 mm in diameter or less) and have sharp curves, b) spinal arteries supplying the fistula have many important side branches and c) the veins supplying the fistulas form collateral pathways if not all collateral pathways supplying the fistula are casted out with embolic agent.

By accidentally embolizing side branches when embolic agent refluxes, the operator can cause tetraplegia, i.e., permanent paresis of the legs, arms, and even death due to paresis of the diaphragm/breathing muscles. The present disclosure as described herein can prevent such side effects by effectively sealing off the catheter tip, and at the same time small and flexible enough to fit into the tiny and highly curvy arteries of the spinal cord.

As such, the catheter of the present disclosure can help to permanently cure the fistula, because by sealing off the catheter tip, the embolic agent can be injected with higher pressure and therefore better penetrate into all collateral pathways supplying the fistula.

Similar situations can be seen in the brain for example, there can be a dural fistula in the floor of the anterior cranial fossa that is supplies by a branch of the ophthalmic artery (this artery also supplies the eye). To be able to cure the fistula, it is important to access the branch of the ophthalmic artery and inject the liquid embolic agent without any reflux so as to affect the supply to the eye.

### Experimental

Three randomized controlled trials were performed that compared middle meningeal artery embolization (MMAE) in addition to standard of care vs. standard of care in patients with non-acute subdural hematoma (SDH). All three trials showed a significant benefit of MMAE compared to standard of care with no increase in complications.

The MAGIC-MT trial was conducted in China and randomized 727 patients to either best medical care, which included medical management and/or surgical treatment, or MMAE with Onyx in addition to best medical care. The primary endpoint, which was any death, symptomatic SDH progression or recurrence, was seen in 7.2% in the MMAE arm vs. 12.2% in the control arm, corresponding to an odds ratio of −4.93 (95% confidence interval −9.37-0.063).

Serious adverse events were significantly less common in the MMAE arm (6.7%) vs. the control arm (11.6%), p=0.02.

The STEM trial was conducted in the US and randomized 310 patients with non-acute SDH to either MMAE using SQUID™ in addition to standard of care or standard of care alone. The primary endpoint, which was residual/recurrent SDH or any new major disabling stroke, myocardial infarction or death from any neurological cause, was observed in 15.2% in the MMAE arm and 39.2% in the control arm, suggesting a significant benefit of MMAE with an odds ratio of 3.60 (95% confidence interval 1.91-6.78).

The EMBOLISE trial was conducted in the US and randomized 400 patients to either best medical care, or MMAE with Onyx in addition to best medical care. The primary endpoint, which was SDH recurrence/progression requiring surgical drainage, occurred in 4.1% in the MMAE arm and 11.3% in the control arm, suggesting a significant benefit of MMAE with a relative risk of 0.36 (95% confidence interval 0.11-0.80).

1. A catheter comprising:
a micro catheter body having a distal catheter tip for placement within a middle meningeal artery (MMA), the catheter body configured with a sealing system adjacent the catheter tip, the sealing system being non-inflatable configured to enable contact with an MMA vessel wall to effect sealing of distal fluids from proximal fluids on opposite sides of the sealing system and wherein the sealing system has sufficient compressibility to enable flexure of the sealing system around a tight bend of a patient's vasculature and to enable passage of an embolizing agent through the catheter body and from the distal catheter tip.

2. The catheter of claim 1 wherein the sealing system includes a bulb having a bulb body, the bulb body having tapered distal surface.

3. The catheter of claim 2 wherein the sealing system includes a bulb having a bulb body, the bulb body having tapered proximal surface.

4. The catheter as in claim 1 where the sealing system is a truncated bulb having a bulb body having a tapered distal surface and a truncated proximal surface.

5. The catheter of claim 1, wherein the bulb body includes one or more slits, slots or channels.

6. The catheter as in claim 1 where the bulb body includes at least one circumferential, longitudinal or helical slit, slot or groove.

7. The catheter as in claim 1 where the sealing system includes an invertable conical leaf having a stiffness enabling proximal inversion as the catheter body is distally advanced within a vessel and distal inversion when the catheter is moved proximally within a vessel.

8. The catheter as in claim 1 where the sealing system includes at least one outwardly extending disk from the catheter body.

9. The catheter as in claim 1 where the sealing system has an outer diameter of 0.8-1.7 mm.

10. The catheter as in claim 1 where the catheter body has an outer diameter of 0.5-1.0 mm.

11. The catheter as in claim 1 wherein the sealing system is 0-50 mm from the distal catheter tip.

12. The catheter as in claim 11 wherein the sealing system is 0-5 mm from the distal catheter tip.

13. The catheter as in claim 1 wherein the outer diameter of the sealing system is 1.5-2.5 times the OD of the catheter body.

14. The catheter as in claim 1 having a radio-opaque marker.

15. A kit comprising at least two catheters of claim 1 wherein each catheter has a sealing system having a different outside diameter.

16. A method of manufacturing a catheter as described in claim 1 comprising the steps of sliding a sealing system having an internal diameter appropriately sized to an outer diameter of the catheter body and gluing the sealing system to the catheter body.
